(19) United States
(12) Patent Application Publication
(10) Pub. No.: US 2022/0273921 A1
(43) Pub. Date: Sep. 1, 2022

(54) STEM CELL SKIN KIT FOR AT-HOME SELF-APPLICATION

(71) Applicant: Stem Cell Revive LLC, Las Vegas, NV (US)

(72) Inventors: Joseph Tracy, Las Vegas, NV (US); Majara Belisle, Las Vegas, NV (US); Mark Meyer, Durango, CO (US)

(21) Appl. No.: 17/187,687

(22) Filed: Feb. 26, 2021

(51) Int. Cl. A61M 35/00; A61M 5/19; A61K 31/728; A61K 35/28; A61K 35/50; A61K 9/00

(52) U.S. Cl. CPC A61M 35/003; A61M 5/19; A61K 31/728; A61K 35/28; A61K 35/50; A61K 9/0014

(57) ABSTRACT

A method of self-administration of growth factors, polypeptides and other therapeutic agents, derived from pluripotent stem cells, via a non-invasive application, and formulated and offered as a kit for reduced cost, improved storage, increased ease, and increased convenience of practitioner, partner, care assistant, or end-user administration.

### CROSS-REFERENCE TO RELATED APPLICATION

The present application is related to U.S. Provisional Patent Application No. 63/983,525, filed on Feb. 28, 2020, which is hereby incorporated in its entirety.

### FIELD OF THE INVENTION

The present invention relates to methods, devices, and device kits for non-invasive topical application, including end-user self-application, of therapeutic growth factors, cytokines, interleukins, peptides, carbohydrates, extracellular matrix proteins, amino acids, and other related therapeutic and cosmetic agents for cosmetic skin applications, administration, and treatments.

### BACKGROUND OF THE INVENTION

Men and women around the world spend billions of dollars annually to cosmetically, surgically or pharmaceutically remediate real and/or perceived physical imperfections such as hair loss, obesity, skin irregularities, poor complexions, aging and body aesthetics (as exemplified by substantial prevalence of augmentation surgery). A wide range of both regulated and unregulated treatments are available to the public. These treatments range from simple over-the-counter solutions such as ointments, lotions, creams and pills to complex procedures that can be administered only by licensed professionals such as aestheticians, physicians, etc. Treatment efficacies and costs vary significantly, as do the degrees of physical invasiveness, pain, discomfort and recovery times. There is a present need for innovative approaches to non-invasive cosmetic treatment that provide simplicity, user convenience and reduced recovery time, while promoting therapeutic effectiveness at a reduced cost. It is especially important in a situations of increased public health risk, such as worldwide pandemic, that professional-level treatments can be safely administered by a partner or self-administered in a user's own home.

### SUMMARY OF THE INVENTION

The present method includes a method and kit for self-administration (or practitioner administration or partner administration) of an acellular liquid allograft solution containing human growth factors derived from multipotent stem cells (for example, human mesenchymal stromal cells (hMSC) and human amnion epithelial cells (hAEC), as are found in such sources as, but not limited to, amniotic fluid and placental tissue. The stem cell-derived fluid containing the growth factors may be fortified and/or combined with other therapeutic agents, including but not limited to peptides, polypeptides, vitamins, minerals and hyaluronic acid. The solution administration is via a non-invasive application, and formulated and offered as a kit for reduced cost, increased ease, and increased convenience of practitioner application, application by partner, or end-user self-application, including at-home use.

The kit of the present invention comprises a dual-syringe mixing system. In a preferred embodiment, each kit contains two syringes connected by a closed valve. One syringe contains a liquid allograft solution of human growth factors. The other syringe contains a hyaluronic acid solution that may be supplemented or fortified with polypeptides. These therapeutic components are thus stored and maintained separately. When ready to apply, practitioners, partners, or end-users act to open the valve and then mix the solutions, by gently moving the syringe plungers in a back-and-forth fashion until a relatively homogeneous and stable mixture is achieved. In the next step of the preferred method of the invention, the user will transfer the mixed solution into a nano infusion pen cartridge. Upon loading of the therapeutic agent mixture, the loaded nano cartridge is then inserted into a nano infusion pen for administration. The dual-syringe mixing system is generally a single-use system, and may be disposed of after use.

The stem cell-derived liquid allograft of the invention contains no live stem cells. The allograft solution is derived from stem cells that have been harvested from non-embryonic placental and/or amniotic fluids. When cultured, such stem cells produce a wide array of active, natural agents, from which the present allograft is sourced and purified to produce the allograft of the invention, such natural agents including: human growth factors, including but not limited to bFGF, epidermal growth factor (EGF), vascular endothelial growth factor (VEGF), transforming growth factor-beta (TGF-β), interleukin-10 (IL-10). The liquid allograft, or skin serum, may also contain cytokines, amino acids, carbohydrates, and extracellular matrix proteins, along with such other ingredients as water, sodium hyaluronate (hyaluronic acid), potassium sorbate and citric acid. Subsequent to culturing, all live stem cells are removed, leaving only the derivative allograft solution of growth factors remaining.

The hyaluronic acid (sodium hyaluronate) component of the present invention is a substance found naturally occurring in the skin. Hyaluronic acid facilitates the binding of water to collagen and serves as a common ingredient in many commercially available moisturizers. The hyaluronic acid may be fortified with polypeptides.

The kit of the invention will provide and include two nano cartridges that serve to hold the liquid allograft/hyaluronic acid therapeutic mixture, or skin serum. Each cartridge has a fine mesh surface on one end. In a method of the invention, practitioners or end-users will fill the nano cartridge with the therapeutic mixture and then insert the nano cartridge into a nano infusion pen. The nano cartridges are packaged in individually sealed wrappers are intended for single-use, to be disposed of after use.

The nano infusion pen component of the invention is a small, hand-held tool that serves to hold and oscillate a nano cartridge that is filled with the therapeutic agent mixture. In a preferred embodiment, the nano infusion pen has four (4) variable speeds for application use, is rechargeable and reusable across multiples uses. Depending on the speed freely selected by the practitioner, partner, or end-user, the nano infusion pen oscillates the nano cartridge head at a rate of 3000-5500 rpm. In a preferred method and use of the invention, the nano infusion pen is set by the user to oscillate at a 5500 rpm (speed: 4) setting.

As an embodiment of the method of the invention, as the nano infusion pen oscillates the nano cartridge head at a rate of 3000-5500 rpm, the nano cartridge's mesh end gently abrades, but does not penetrate, the end-user's skin, upon contact, to create microscopic channels. Simultaneous with such abrasion, the nano cartridge releases and applies the therapeutic mixture directly to the area(s) of skin being treated. The tiny epidermal channels created by the abrasion promote fast and deep penetration of the liquid allograft solution. The therapeutic agents in the liquid allograft (e.g., growth factors, cytokines, amino acids) possess significant therapeutic benefits when used as a treatment to stimulate tissue repair and regeneration, and to stimulate beneficial collagen and elastin production. Even in the absence of the liquid allograft, the abrading action of the pen and cartridge confers health benefits, as the skin's subsequent healing processes serve to stimulate the beneficial production of collagen and elastin.

Application and oscillating action of the cartridge's mesh end upon the end-user's skin surface does not penetrate the skin, and thereby the therapeutic mixture is applied topically. Consequently, the method of the invention constitutes a non-invasive procedure that does not require a licensed professional such as a doctor or aesthetician to administer and thus finds greater and broader application than previous methods found in the art.

In a preferred embodiment and method of the invention, a kit may contain a 30-day supply of hyaluronic acid to be applied daily to the skin following initial application of the liquid allograft treatment with the therapeutic mixture via nano pen. The hyaluronic acid moisturizes and facilitates healing processes of the skin. Application of the pure hyaluronic acid is by hand, either by the end-user, by a partner, or by an attendant. In a preferred embodiment of the invention, and to reduce the likelihood of bacterial contamination, the hyaluronic acid is packaged in separate, single-use 1 ml ampoules that may be safely disposed of after use.

Polypeptides are linear organic polymers comprising large numbers of amino-acid residues bonded together in chains. Select polypeptides therapeutically that are useful for the present invention may include polypeptides derived from marine plant sources and recognized to help build cells and collagen. In a preferred embodiment of the invention, a polypeptide treatment is delivered in a dual syringe mixing system provided in a kit corresponding to the liquid allograft/hyaluronic acid therapeutic mixture administration kit detailed above. For the polypeptide treatment, one syringe contains the polypeptide solution; the other contains hyaluronic acid. When ready for use and application, practitioners or end-users open the valve and mix the solutions by moving the syringe plungers in a back-and-forth manner. The mixed solution is loaded into a nano pen cartridge, which is then loaded into the nano infusion pen, as per above.

In a preferred alternate embodiment, the polypeptide solution may be modified by fortifying it with other nutrient rich materials, including vitamins, minerals, other peptides, and/or cannabidiol and related agents.

### BRIEF DESCRIPTION OF THE DRAWINGS

In the accompanying drawings which form a part of the specification and are to be read in conjunction therewith, and in which like reference numerals are employed to indicate like parts in the various figures:

FIG. 1 is a perspective view of a therapeutic agent kit of the present invention, according to an exemplary embodiment, including a nano infusion pen, nano cartridges, quantities of the liquid allograft “skin serum” and quantities of hyaluronic acid essence, provided in a container box for convenience and ease of shipment and storage.

FIG. 2 is a perspective view of a therapeutic agent kit of the present invention, according to an exemplary embodiment, including a nano infusion pen, a disposable dual mixing syringe apparatus with connecting valve and separated compartments for liquid allograft and hyaluronic acid therapeutic agents, quantities of the liquid allograft “skin serum” and quantities of hyaluronic acid essence, provided in a container box for convenience and ease of shipment and storage.

FIG. 3 is a perspective view of a therapeutic hyaluronic essence (acid) (monthly supply) kit of the present invention, according to an exemplary embodiment, including a dual mixing syringe apparatus with connecting valve and separated compartments for liquid allograft and hyaluronic acid therapeutic agents, quantities of the liquid allograft “skin serum” and quantities of hyaluronic acid essence, provided in a container box for convenience and ease of shipment and storage.

FIG. 4 is a perspective view of a nano infusion pen of the present invention, along with two annular plastic end cap guards for individual fixture to the abrading mesh surface end of the pen for use during operation, according to an exemplary embodiment, depicted as provided in a container box for convenience and ease of shipment and storage.

FIG. 5 is a perspective view of a nano infusion pen of the present invention according to an exemplary embodiment.

FIG. 6 provides a perspective view of a nano infusion pen cartridge abrading mesh surface end of the present invention according to an exemplary embodiment.

FIG. 7 is a perspective view of a dual mixing syringe apparatus of the invention, according to an exemplary embodiment, featuring manually operable connecting valve and separated compartments/volumes for liquid allograft and hyaluronic acid therapeutic agents.

FIG. 8 is a perspective view of a 30-day supply of hyaluronic acid (daily) “aftercare” treatment ampoules of the present invention according to an exemplary embodiment.

FIG. 9 is a perspective view of a single-use treatment quantity of hyaluronic acid in an ampoule suitable for (daily) “aftercare” treatment and ease of storage and disposal, per the present invention and according to an exemplary embodiment.

These components in the figures are not necessarily to scale, emphasis instead being placed upon illustrating the principles of the invention. In the figures, identical reference numerals may designate corresponding parts throughout the different views.

### DETAILED DESCRIPTION OF THE INVENTION

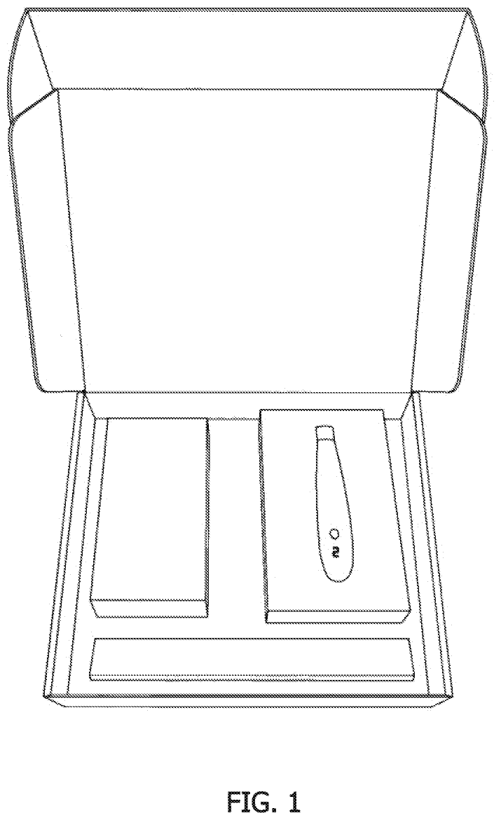

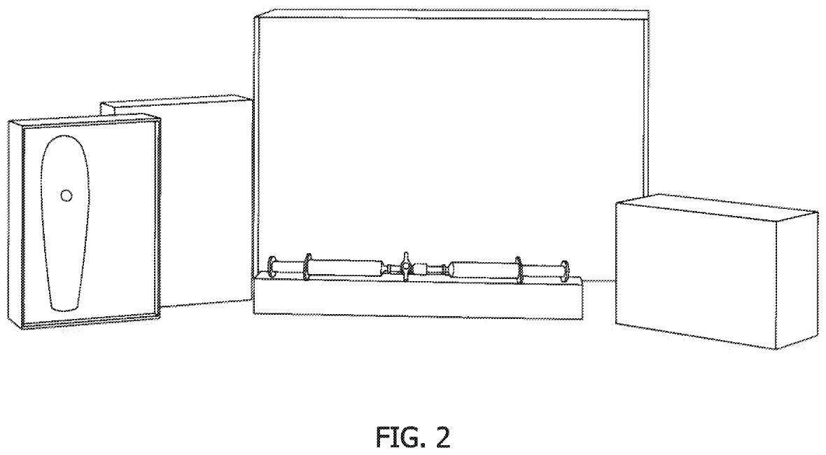

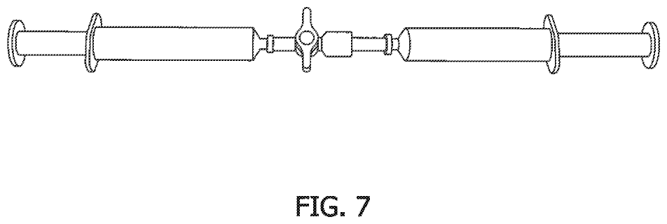

FIG. 1 and FIG. 2 are perspective views of exemplary embodiments of the present invention. As depicted, the Stem Cell Skin Kit for At-Home Self-Application contains all materials required for a simple, easy do-it-yourself at-home application of a therapeutic agent mixture that can be administered by a medical practitioner, other licensed professional (e.g., aesthetician), an attendant, a partner, or self-administered by an end-user and completed in 30-45 minutes. The kit comprises a reusable nano infusion therapeutic pen and disposable nano cartridges for administration of a therapeutic agent mixture, a disposable dual mixing syringe apparatus with manually operable connecting valve and separate syringe compartments/volumes for stem cell-derived liquid allograft “skin serum” therapeutic agent and for hyaluronic acid essence optionally fortified with polypeptides, assembled together in a single container box that is suitable and promotes convenience and ease of use, shipment and storage. As illustrated in FIG. 7, in a preferred embodiment, the disposable dual mixing syringe comprises two 5.0 ml syringes connected by a manually operable closed connecting valve, wherein one syringe is suitable for containing and delivering 0.5 ml of the liquid allograft and the other syringe is suitable for containing and delivering 4.5 ml hyaluronic acid, optionally fortified with polypeptides. In a preferred method and preferred use of the present invention, when ready to start the skin treatment, the health practitioner or end-user opens the valve connecting the dual syringes and mixes the stem cell derivative liquid allograft and hyaluronic acid (optionally fortified with polypeptides) solutions by gently pushing the syringe plungers back and forth 3-4 times. After mixing, the entire 5.0 ml therapeutic agent mixture solution is “stored” in one syringe until time for administration via direct application to the skin treatment area.

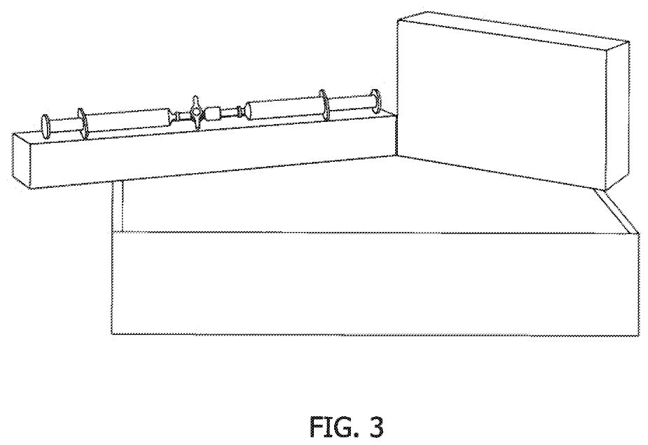

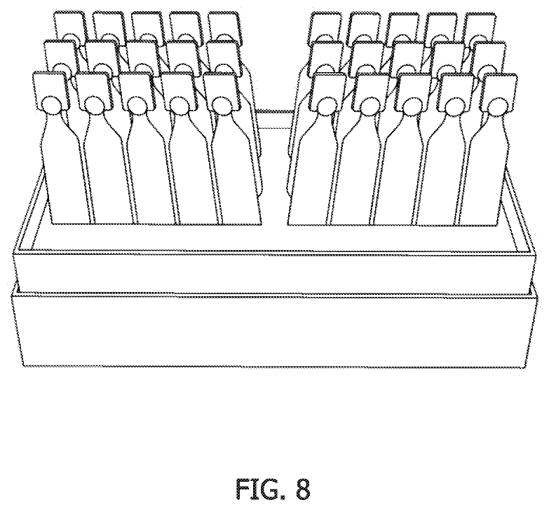

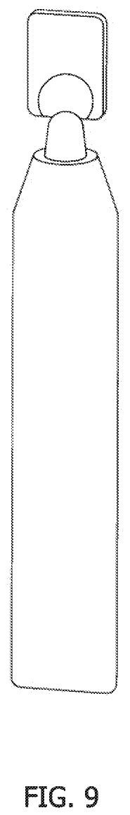

FIG. 3 is a perspective view of an exemplary embodiment of the present invention. In a preferred embodiment, a kit of the present invention provides a monthly supply, including proper quantities of stem cell-derived liquid allograft “skin serum” and quantities of hyaluronic acid essence fortified with polypeptides. As illustrated in FIG. 8, in a particular preferred embodiment and method of the invention, a kit may contain a 30-day supply of pure hyaluronic acid to be applied daily to the skin, following initial treatment with the therapeutic agent mixture via nano infusion pen and nano mesh cartridge. The pure hyaluronic acid hydrates the skin facilitates natural healing processes of the skin from the microscopic channels created by the nano infusion pen during liquid allograft application, for a supple, healthy appearance. Application of the pure hyaluronic acid is by hand. In a preferred method and use of the invention, daily manual application of the hyaluronic acid is performed prior to bedtime. In another preferred method and use of the invention, the hyaluronic acid application is allowed by the user to fully absorb for approximately 10-15 minutes. As depicted in FIG. 9, in a preferred embodiment of the invention, and to reduce the likelihood of bacterial contamination, the hyaluronic acid is packaged in separate, single-use 1 ml ampoules that may be safely disposed of after use. In other preferred embodiments, the kit is suitable for providing supplies of the kit components for shorter or longer therapeutic regimens, including a weekly supply, a two-month supply, a quarterly (three month) supply, a six-month supply, or supplies for other shorter or longer effective therapeutic regimen periods. In a preferred method and use of the invention, the intensive stem cell-derived liquid allograft “skin serum” is applied once every six months as part of an optimal skin care regimen. In another preferred embodiment, a supply for monthly applications of polypeptide-fortified hyaluronic acid may be included with the kit.

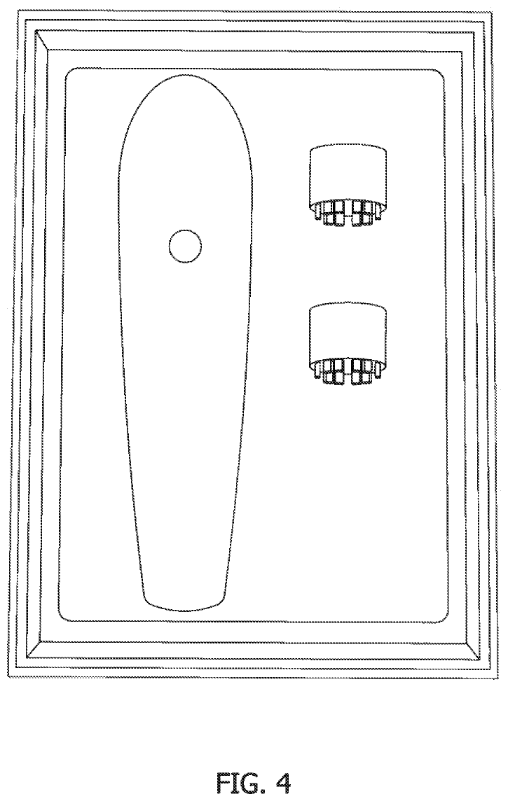

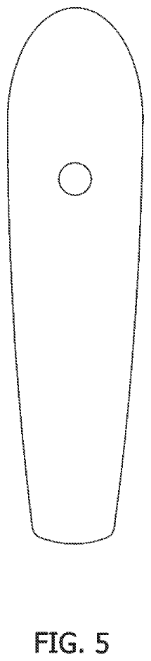

FIG. 4 and FIG. 5 are perspective views of an exemplary embodiments of a nano infusion pen of the present invention. The nano infusion pen is a small, hand-held tool that serves to hold and oscillate the nano mesh cartridge filled with the stem cell-derived liquid allograft “skin serum” therapeutic agent and hyaluronic acid with polypeptides therapeutic mixture. In FIG. 4, perspective views of annular end guards of the present invention, for single individual attachment to secure and protect the nano pen cartridge (with abrading mesh surface) within the open end of the nano infusion pen prior to and during operation, are presented. In a preferred embodiment, the nano infusion pen has four (4) variable speeds for application use, is rechargeable and reusable across multiple treatment administrations. Depending on the speed freely selected and adjusted by the practitioner or end-user, the nano infusion pen oscillates the nano mesh cartridge head at a rate of 3000-5500 rpm. Substantively, the nano infusion pen is the tool that allows the nano mesh cartridge to a) prepare the skin to receive and efficiently absorb the stem cell derivative liquid allograft and hyaluronic acid (with polypeptides) solution and b) distribute the solution simultaneously. In a preferred method of the invention, using gentle pressure, users simply rub the nano infusion pen in small circular motions on the desired treatment areas of the skin of the face, neck, chest, or other areas of the body to gently abrade the skin and simultaneously, topically apply the stem cell-derived liquid allograft and hyaluronic acid (with polypeptides) solution. In a particular embodiment of the method, when empty, users may refill the nano mesh cartridge with any remaining solution, which may be used to complete the application or to concentrate on preferred target areas or personal trouble spots.

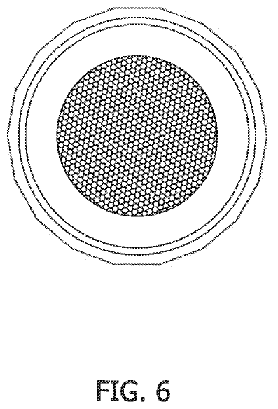

FIG. 6 is a perspective view of a mesh end of a nano infusion pen cartridge according to a preferred embodiment of the invention. In a preferred embodiment of the invention, each Stem Cell Skin Kit for At-Home Self-Application includes two individually wrapped 3.0 ml empty nano mesh cartridges. One end of each cartridge has a mesh surface that, when activated for therapeutic agent administration, the nano infusion pen oscillates the nano mesh cartridge to thereby gently abrade the skin, while simultaneously applying and delivering the stem cell-derivative liquid allograft/hyaluronic acid (with polypeptides) therapeutic agent mixture solution.

In a preferred method of the present invention, the small epidermal channels created by the abrasion of the skin of the treatment area encourage fast and deep penetration of the therapeutic agent mixture solution. In another embodiment of the method, upon the abrasion of the skin, even in the absence of any therapeutic agent mixture solution, the skin's subsequent healing stimulates the natural, beneficial production of collagen and elastin. In preferred embodiments of the invention, the nano mesh cartridge's mesh end does not penetrate the skin and the therapeutic agent mixture solution is thereby applied topically. In a preferred method of the invention, the process and administration of a therapeutic agent mixture solution is a non-invasive procedure that does not require a licensed professional, such as a doctor or aesthetician, to administer and is suitable for an end-user individual to self-administer and self-treat. In another preferred method, the administration may be by a partner or by a care assistant. In an alternate preferred method, the process and administration of a therapeutic agent mixture solution may be conducted by licensed professionals using microdermabrasion or dermabrasion techniques.

In another preferred embodiment of the invention, a polypeptide treatment is delivered in a disposable dual mixing syringe apparatus provided in a kit corresponding to the stem cell-derivative liquid allograft and hyaluronic acid (with polypeptides) therapeutic mixture administration kit detailed previously. For the polypeptide treatment, one syringe contains the polypeptide solution; the other contains hyaluronic acid (optionally also with polypeptides). When ready for use and application, practitioners or end-users open the valve and mix the solutions by moving the syringe plungers in a back-and-forth manner. The mixed solution is loaded into a nano infusion pen mesh cartridge, which is then loaded into the nano infusion pen, as per above. In a specific, preferred embodiment of the invention, the “polypeptide skin serum” of the invention includes deionized water, sodium hyaluronate (hyaluronic acid) (NASHA grade), aloe barbadnesis (aloe leaf) gel, vegetable glycerine, Cucumis sativus (cucumber) fruit extract, chrysin, Pentavitin®, palmitoyl oligopeptide, n-hydroxysuccinimide, palmitoyl tetrapeptide-3, palmitoyl pentapeptide-3, steareth-20, polysorbate-20, carbomer, phenoxythanol, caprylyl glycol (Optiphen), potassium sorbate, and citric acid-20. In a particular preferred embodiment, the polypeptide treatment may be applied on a monthly basis during the treatment regimen.

In a preferred alternate embodiment, the polypeptide solution may be modified by fortifying it with other nutrient-rich materials, including but not limited to, vitamins, minerals, other peptides, and/or cannabidiol and related therapeutic agents.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A kit for non-invasive topical application of a mixture in solution, comprising:
a dual-chamber mixing syringe comprising an internal chamber containing a liquid allograft solution and a separate internal chamber containing a hyaluronic acid-based solution, wherein the mixing syringe further comprises a valve to alternately separate the internal chambers for initial solution storage and to allow the chamber content solution to fully mix prior to mixture administration;
a solution cartridge with one of more mildly abrasive mesh external tips, further comprising a receptacle suitable for receiving the allograft solution and hyaluronic acid-based solution mixture;
an electric nano-infusion pen delivery device suitable for hand-held user control, further comprising an operable oscillating mechanism, further wherein the solution cartridge with mesh tip may be operably inserted into the pen delivery device suitably for hand-held manual topical administration of the solution mixture; and
one or more ampoules of hyaluronic acid solution for periodic adjunct therapeutic solution application.

2. The kit of claim 1, wherein the liquid allograft solution further comprises polypeptides.

3. The kit of claim 1, wherein the kit further comprises active topical agents for optional application.

4. A method of non-invasive topical application of a mixture in solution via a delivery device, comprising:
operating a valve between chambers of a dual-chamber mixing syringe comprising an internal chamber containing a liquid allograft solution and a separate internal chamber containing a hyaluronic acid-based solution, wherein the mixing syringe comprises such valve to alternately separate the internal chambers for initial solution storage, and to allow the chamber content solution to fully mix for administration;
decanting the mixed solution into a solution cartridge with one or more mildly abrasive mesh external end tips, further comprising a receptacle suitable for receiving the allograft solution and hyaluronic acid-based solution mixture;
inserting the solution cartridge with one or more mesh external end tips into an electric nano-infusion pen delivery device suitable for hand-held user control, the pen delivery device further comprising an operable oscillating mechanism, further wherein the solution cartridge with mesh external tip is placed such that the tip is distal to the pen delivery device; and
operating the pen delivery device oscillating mechanism such that the solution mesh cartridge oscillates as the solution mixture is effectively administered topically to a subject.
